(19) United States
(12) Patent Application Publication
(10) Pub. No.: US 2026/0224805 A1
(43) Pub. Date: Aug. 6, 2026

(54) INTRATHECAL DRUG DELIVERY SYSTEM

(71) Applicant: ASPIRE BIOMED LLC, Edison, NJ (US)

(72) Inventors: Divya Gupta, Edison, NJ (US); Pankaj Gupta, Edison, NJ (US); Karishma Gupta, Edison, NJ (US); Saurin Shah, Woodbury, MN (US)

(21) Appl. No.: 19/415,147

(22) Filed: Dec. 10, 2025

Related U.S. Application Data
(60) Provisional application No. 63/754,763, filed on Feb. 6, 2025.

(51) Int. Cl. A61M 5/142; A61M 5/168; A61M 25/00; A61M 39/00; A61M 39/02; A61M 39/04

(52) U.S. Cl. CPC A61M 5/14276; A61M 5/16827; A61M 25/0026; A61M 39/0208; A61M 39/04; A61M 2039/0072; A61M 2039/0211; A61M 2039/0238; A61M 2202/0464; A61M 2205/18; A61M 2205/32; A61M 2205/3303; A61M 2205/3313; A61M 2205/3368; A61M 2205/3523; A61M 2205/702; A61M 2205/8206

(57) ABSTRACT

The present invention relates to an implantable intrathecal drug-delivery system (1000) includes a subcutaneously implantable housing (102) coupled to a multi-lumen catheter (130) positioned to access the intrathecal space. The housing contains drug reservoirs (110a-110n), a pump (150), an optional CSF reservoir (120), one or more biosensors (160) configured to obtain from CSF a signal indicative of therapeutic-agent concentration, a processor (170) operatively coupled to the biosensor(s) and the pump, a wireless module (180), and a transdermal access port (190). Based on biosensor-derived concentration data, the processor determines the concentration in real time and adjusts infusion parameters to maintain a target range. The catheter can include lumens for CSF inflow (134), CSF return (136), drug delivery (138), and power/data (140); the biosensor(s) can include near-infrared spectroscopy and auxiliary sensors. Methods provide CSF monitoring, automatic or programmatic infusion adjustment, and secure refill and CSF sampling.

### CROSS REFERENCE TO RELATED APPLICATIONS

This application claims priority of U.S. Provisional Application No. 63/754,763 filed on Feb. 6, 2025 under 35 U.S.C. § 119(e), the entire contents of which are hereby incorporated by reference.

### FIELD OF THE INVENTION

The present invention relates to the field of medical devices, and more specifically to a drug delivery system for the precise administration of drugs into the cerebrospinal fluid (CSF).

### BACKGROUND

The management of medical conditions such as pain, neurological and psychiatric disorders often require the targeted delivery of therapeutic agents to the central nervous system (CNS). Such conditions encompass a wide range of diseases, including but not limited to chronic and caner pain, spasticity (e.g., cerebral palsy, spinal cord injury, multiple sclerosis), Parkinson's disease, seizure disorders, narcolepsy, brain tumors, bipolar disorder, neuro-inflammatory and infectious conditions (e.g., meningitis where intrathecal antimicrobials may be indicated), post-operative analgesia, and peri-parturient/obstetric scenarios and various forms of chronic pain. Traditionally, these medications are administered via oral or intravenous routes. However, these delivery methods are accompanied by several limitations and challenges that hinder their efficacy in treating CNS conditions.

One significant issue with conventional drug delivery is the fluctuation in drug concentration within the cerebrospinal fluid (CSF) between successive doses. This variability can result in the “on-off” phenomenon, where the therapeutic effect of the drug is either too strong or wears off prematurely, creating a cycle of ineffective symptom management. Such fluctuations are particularly problematic in conditions like Parkinson's disease, which require stable and continuous drug levels to effectively control symptoms. Furthermore, oral drug administration presents challenges for subjects who require complex dosing regimens involving multiple drugs or multiple doses throughout the day. This issue is further exacerbated in subjects with conditions such as swallowing difficulties, neurological impairments, or cognitive disorders, which often lead to poor medication adherence and suboptimal therapeutic outcomes.

Oral medications must be administered in higher doses to overcome the drop in drug concentration caused by first-pass metabolism in the liver and further reduction after the drug crosses the blood-brain barrier (BBB). The BBB serves as a semi-permeable membrane that protects the brain and spinal cord by regulating the movement of molecules and ions between the blood and the CNS. While this mechanism is vital for protecting the brain from harmful substances, it also limits the effectiveness of orally administered drugs by reducing their bioavailability within the CNS. This necessitates higher drug doses, which can lead to increased treatment costs and a heightened risk of systemic side effects.

Systemic side effects associated with CNS drugs pose a significant challenge in subject management, as they can impair the overall health of the subject and reduce their quality of life. These side effects can be severe, and in some cases, life-threatening. Moreover, when CNS drugs are administered to pregnant subjects, concerns arise regarding fetal exposure to these medications, which may result in developmental issues and long-term health consequences for the child. In contrast, intrathecal drug delivery directly administers therapeutic agents into the CSF, bypassing the BBB and reducing drug exposure to the bloodstream and other organs, thereby minimizing systemic side effects and enhancing the targeted delivery of the medication.

Despite these advantages, conventional intrathecal drug delivery systems, such as intrathecal pumps, still face several critical limitations. These systems typically operate at a predefined, fixed infusion rate and do not adapt to fluctuations in CSF drug concentrations. This necessitates regular clinical monitoring and manual adjustments, which can be cumbersome and inconvenient for both subjects and healthcare providers. Additionally, current intrathecal drug delivery systems require the subject to be present in close proximity for adjustments, limiting their flexibility and convenience. The lack of remote management capabilities further restricts their effectiveness in managing chronic conditions.

Moreover, the existing systems are generally designed to accommodate only a narrow range of drugs and are primarily used for specific clinical conditions, such as chronic severe pain or end-stage cancer. This limited applicability restricts the potential use of these systems in broader subject populations, particularly those with other neurological or psychiatric conditions requiring targeted, continuous drug delivery and long-term treatment.

The aforementioned limitations highlight the need for an advanced intrathecal drug delivery system capable of dynamically adjusting to fluctuating drug concentrations, offering remote management capabilities, and accommodating a wider range of therapeutic agents and clinical indications.

### SUMMARY

This summary is neither intended to identify essential features of the present invention nor is it intended for use in determining or limiting the scope of the present invention.

The present disclosure provides implantable systems, devices, methods, for closed-loop intrathecal drug delivery. In one aspect, an implantable system determines, in real time, a drug concentration in cerebrospinal fluid (CSF) and automatically adjusts infusion within predefined safety bounds.

In some embodiments, the system provides in-situ CSF sensing at or near the intrathecal space, for example, using near-infrared (NIR) spectroscopy, and optionally additional sensors (e.g., pressure, temperature, osmolarity, or biomarker sensors).

In some embodiments, the system supports multi-agent therapy via multiple independently addressable reservoirs with microvalves configured to enable sequential, alternating, or simultaneous administration according to programmable dosing schedules.

In some embodiments, a multi-lumen catheter architecture partitions CSF sampling/return from drug delivery. It may incorporate an optical window to facilitate spectroscopic sensing while reducing cross-contamination.

In some embodiments, a CSF reservoir within the device housing enables therapeutic drug monitoring, calibration to patient-specific baselines, and optional diagnostic workflows.

In some embodiments, authenticated wireless communication provides telemetry, clinician programming, and optional synchronisation with other implanted devices or external therapeutic management platforms.

In some embodiments, patient-notification functionality (e.g., visual, auditory, or haptic output) indicates therapy events, refill windows, and device faults.

In some embodiments, transdermal refilling and CSF sampling are provided via an access port having a self-sealing septum and localisation features (e.g., radiopaque and/or wireless locator elements).

In some embodiments, processor-enforced safety features include hard and soft infusion-rate limits, occlusion and fault detection, signal-quality monitoring, and automatic suspension or fallback modes upon detection of out-of-range conditions.

Additional aspects include computer-implemented methods for operating the closed-loop system and non-transitory computer-readable media storing instructions that, when executed by one or more processors, cause performance of operations including receiving CSF sensor data, estimating drug concentration, and adjusting infusion parameters according to programmable constraints.

The present invention provides an intrathecal drug delivery system that is responsive to the drug concentrations in the CSF and may optimize the administration of central nervous system (CNS) acting drugs for various common diseases and can be used for long term maintenance therapy.

In an aspect of the present invention, the intrathecal drug delivery system comprises an implantable pump, a multi-lumen catheter, one or more sensors or biosensors, plurality of reservoirs for drugs and/or CSF, an electronic control module, a battery module, a transcutaneous access port for injecting a drug refill or for aspirating a CSF sample, and a wireless communication module. The sensors may continuously monitor the CSF drug concentration, providing real-time data based on which the drug infusion rates may be adjusted, thereby optimizing therapeutic efficacy. The multi-lumen catheter allows concurrent administration of multiple drugs if required while allowing for precise monitoring and infusion control. Wireless communication enables remote monitoring and adjustment of the pump, enhancing treatment flexibility and compliance with subjects. The drug delivery system may help reduce systemic side effects, improve dosing accuracy, enhance subject's quality of life, and reduce healthcare costs.

In another aspect of the present invention, the pump is configured for bidirectional cerebrospinal fluid (CSF) flow, real-time drug concentration monitoring, and automated drug dosing. The pump may be implanted subcutaneously, for example along the flank region or along the spinal column of the subject.

In another aspect of the present invention, the system may comprise a multi-lumen catheter having two, three, or more lumens, extending from the pump housing to the intrathecal space, thereby enabling precise and targeted drug delivery. Preferably, the system comprises a double lumen catheter, wherein, the first lumen may be adapted for CSF inflow from the intrathecal space into the pump housing, and the second lumen may provide CSF and drug outflow from the pump back into the intrathecal space.

In another aspect of the present invention, the system may be configured with at least one sensor to detect and quantify the concentration of drug in the CSF. These sensors provide continuous feedback to the system, enabling dynamic adjustment of drug infusion rates to maintain optimal therapeutic levels. The sensors may be selected from the group comprising a near-infrared spectroscopic sensor, a temperature sensor, a pressure sensor, an environmental sensor, an osmolarity sensor, and a biomarker sensor, or any combination thereof. The pump may have the ability to attach an adjacent extension subcutaneously via an additional lumen for housing the sensors or the reservoirs depending on the number and size of the sensors or the reservoirs.

In another aspect of the present invention, the system may include a plurality of reservoirs, each configured to contain a distinct therapeutic agent. Each reservoir may be equipped with an individual valve controlled by the electronic module to enable the dispensing of drugs. Further, the system may be equipped with an automated module, i.e., when the reservoir reaches a predetermined low level, the system would alert the subject and health care provider, ensuring timely refill and continuous medication availability.

In another aspect of the present invention, the electronic module or a processor may be configured to analyse and calculate the required adjustments to the drug delivery rate, and control the operation of the pump based on feedback from the sensors. Further, the processor may store sensor data, subject treatment regimens and dosing histories to alert the provider of any drug interactions or contraindications. The programming of the processor will include a safety check mechanism to prevent release of a drug in a contraindicated situation unless there is an override by the provider.

In another aspect of the present invention, the wireless communication module uploads data into the provider portal app that facilitates remote monitoring, reporting, and adjustment of the treatment regimen by a healthcare provider, thereby enabling real-time modifications without necessitating the subject's physical presence in a clinical setting. The system may be accessed and controlled remotely via smartphones or similar electronic devices. A patient portal app will also enable to patient to view their treatment regimen and get alerts for drug refills or drug interactions etc.

In another aspect of the present invention, the access port may be adapted for transcutaneous refilling of the drug reservoirs and for sampling CSF fluid when needed. Further, refill ports for each individual reservoir can be located using wireless locator and/or radio opaque marker for precise access for refilling a drug or sampling the CSF fluid when needed. Further, the battery module may be configured to power pump actuation and sensors.

In another aspect of the present invention, the intrathecal pump and catheter may be detachably connected to an auxiliary housing implanted subcutaneously via suture tabs. The auxiliary housing may be configured to accommodate one or more components, including biosensors, drug reservoirs, electronic control module, battery module, and wireless communication module. The intrathecal pump and the auxiliary housing may be positioned on opposite sides of the abdomen and operatively connected via a common multi-lumen catheter. Further, the auxiliary housing may be independently controlled through the wireless communication module.

The system is configured to monitor and adjust the drug delivery based on real-time CSF drug concentrations, thereby optimizing therapeutic efficacy.

In another embodiment of the present invention, the intrathecal drug delivery system may be compatible with therapeutic modalities, such as viral vectors, gene therapies, and antibody therapies.

These and other embodiments, processes, and features of the present invention will become more fully apparent when the following detailed description is read with the accompanying experimental details. However, both the foregoing summary of the present invention and the following detailed description of it represent one potential embodiment and are not restrictive of the present invention or other embodiments of the present invention.

### BRIEF DESCRIPTION OF THE DRAWINGS

It is to be noted, however, that the appended drawings illustrate only typical embodiments of the present subject matter and are therefore not to be considered limiting its scope, as the invention may admit to other equally effective embodiments. The detailed description is described with reference to the accompanying figures. In the figures, a reference number identifies the figure in which the reference number first appears. The same numbers are used throughout the figures to reference like features and components.

FIG. 1 depicting reservoir-to-pump manifolding, CSF sampling/return, and drug infusion via a multi-lumen catheter (130) of the intrathecal system (1000).

FIG. 1A illustrates a block diagram of the intrathecal drug delivery system (1000) having an adjacent extension module (161) for housing biosensors (160, 162) and reservoirs (110a-110n) according to an alternate embodiment of the present invention.

FIG. 2 illustrates a block diagram of the intrathecal drug delivery system (1000) according to an embodiment of the present invention.

FIG. 3 illustrates a cross sectional view of the tip (132) of the multi-lumen catheter (130) according to an embodiment of the present invention.

FIG. 4 illustrates an implantable drug delivery pump (102) placed across the flank region of the patient's body.

FIG. 4A illustrates an implantable drug delivery pump (102) operatively connected to an adjacent extension module (161), placed across the flank region of the patient's body.

FIG. 5 illustrates an implantable device/pump housing (102) and a multi-lumen catheter (130) detachably connected to an auxiliary housing (104) according to an embodiment of the present invention.

FIG. 6 illustrates an implantable drug delivery pump (102) implanted along the abdominal wall of the patient's body.

FIG. 6a illustrates an implantable drug delivery pump (102) and a detachably connected auxiliary housing (104) implanted along the same side of the abdominal wall of the patient's body.

FIG. 6b illustrates an implantable drug delivery pump (102) and a detachably connected auxiliary housing (104) implanted along the opposite sides of the abdominal wall of the patient's body.

FIG. 7 illustrates the tip (132) of the multi-lumen catheter (130) located at the intrathecal space (12).

FIG. 8 illustrates the internal surface (102b) of the implantable device housing (102).

FIG. 9 illustrates a side view of the internal surface (102b) of the implantable device housing (102).

FIG. 10 illustrates the external surface (102a) of the implantable device housing (102).

FIG. 11 illustrates a side view of the external surface (102a) of the implantable device housing (102).

FIG. 12 illustrates the two halves of the device together.

FIG. 13 is an exemplary representation of the tip (132) of the catheter (130) having (a) single, (b) double, (c) triple, and (d) quad lumen.

FIG. 14 is a flowchart depicting closed-loop dosing: measure CSF, evaluate target range, adjust infusion via pump (150) from reservoirs (110a-110n) if needed, then transmit parameters and log trend.

FIG. 15 is a flowchart illustrating secure refill and CSF-sample handling via access port (190) with fail-safe denial on auth failure, optional sensor recalibration (160), and post-procedure logging and restart.

FIG. 16 is a flowchart depicting distal-tip biosensor and housing-mounted variants, each coupled to the catheter tip (132) for CSF sensing.

### CALL OUT LIST

1000 system; 102 device/pump housing; 102a external (skin-facing) side; 102b internal (anatomy-facing) side; 104 auxiliary housing;
110a-110n drug reservoirs; 112a-112n reservoir microvalves;
120 CSF reservoir;
130 multi-lumen catheter; 132 distal tip; 134 CSF inflow lumen; 136 CSF outflow lumen; 138 drug-delivery lumen; 140 data/power lumen (optionally 140a power, 140b data);
130A alternate multi-lumen catheter; 134A alternate CSF inflow; 138A alternate drug-delivery lumen;
150 pump; 160 NIR biosensor; 161 adjacent extension module; 162 additional biosensors (temp/pressure/etc.);
170 processor/controller; 180 wireless module/antenna;
190 access port; 192 self-sealing septum;
200 microfluidic channel; 202 inline particulate filter; 204 check valve (one-way);
206 patient alert (haptic/audible/visual); 210 battery/power source;
12 Intrathecal Space.

The figures depict embodiments of the present subject matter for the purpose of illustration only. A person skilled in the art will easily recognize from the following description that alternative embodiments of the structures and methods illustrated herein may be employed without departing from the principles of the invention described herein.

### DETAILED DESCRIPTION

The following is a detailed description of the present invention as depicted in the accompanying drawings. However, the amount of detail offered is not intended to limit the invention, but rather to cover all modifications, equivalents, and alternatives falling within the scope of the present invention. While aspects of the described systems can be implemented in any number of different configurations, the embodiments are described in the context of the following exemplary system(s). Therefore, specific structural and functional details described herein are not to be interpreted as limiting, but merely as a basis for the claims and as a representative basis for teaching one skilled in the art to variously employ the disclosed concepts in an appropriate structure or method. Furthermore, the terms and phrases used herein are not intended to be limiting, but rather to provide an understandable description of the present invention.

The terminology used herein is for the purpose of describing particular embodiments only and is not intended to be limiting of the invention. As used therein, the term “and/or” includes and all combinations of one or more of the associated listed items. As used therein, the singular forms “a”, “an”, and “the” are intended to include the plural forms as well as the singular forms, unless the context clearly indicates otherwise. It will be further understood that the terms “including”, “having”, “comprises” and/or “comprising”, when used in this specification, specify the presence of stated features, steps, operations, elements, and/or components, but do not preclude the presence or addition of one or more other features, steps, operations, elements, components, and/or groups thereof.

Further, the use of the expression “at least” or “at least one” suggests the use of one or more elements or ingredients or quantities, as the use may be in the embodiment of the disclosure to achieve one or more of the desired objects or results. Furthermore, the term “may” be used herein in a permissive sense (i.e., meaning having the potential to), rather than the mandatory sense, (i.e., meaning must).

The term “therapeutic agent”, “drug”, “medicine” and/or “medication” may be used interchangeably.

As used herein, “intrathecal space (12)” denotes the subarachnoid space containing cerebrospinal fluid (CSF). “In real time” means within the sampling/processing cycle of the device, typically within 1-10 seconds of sensor acquisition, sufficient to inform the next control update. “Biosensor-derived concentration data” means data values or signals produced by one or more biosensors (160) that are indicative of a therapeutic-agent concentration in CSF after optional preprocessing (e.g., baseline correction, normalisation, calibration transform). “Therapeutic-management platform” includes one or more external computing systems (e.g., clinician console, hospital server, or cloud dashboard) configured to receive device telemetry and to send programming/parameter updates to the system (1000). “Wireless communication module (180)” refers to hardware that comprises at least a radio transceiver and antenna, implementing the cited protocols and security primitives.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant prior art and the present disclosure and will be interpreted in an idealized or overly formal sense unless expressly defined herein.

From the present disclosure, it will be understood that a number of features and steps are disclosed. Each of these has individual benefits and each can also be used in conjunction with one or more, or in some cases all, of the other disclosed techniques. Accordingly, for the sake of clarity, this description will refrain from repeating every possible combination of features/components/steps in an unnecessary fashion. Nevertheless, the specification should be read with the understanding that such combinations are entirely within the scope of the invention.

The present invention relates to intrathecal drug delivery systems (1000), implantable pump devices, and methods of drug administration for optimized and personalized treatment of central nervous system (CNS) disorders. The system (1000) is intended for long-term maintenance therapy and supports both bidirectional CSF flow and adaptive drug dosing based on real-time monitoring.

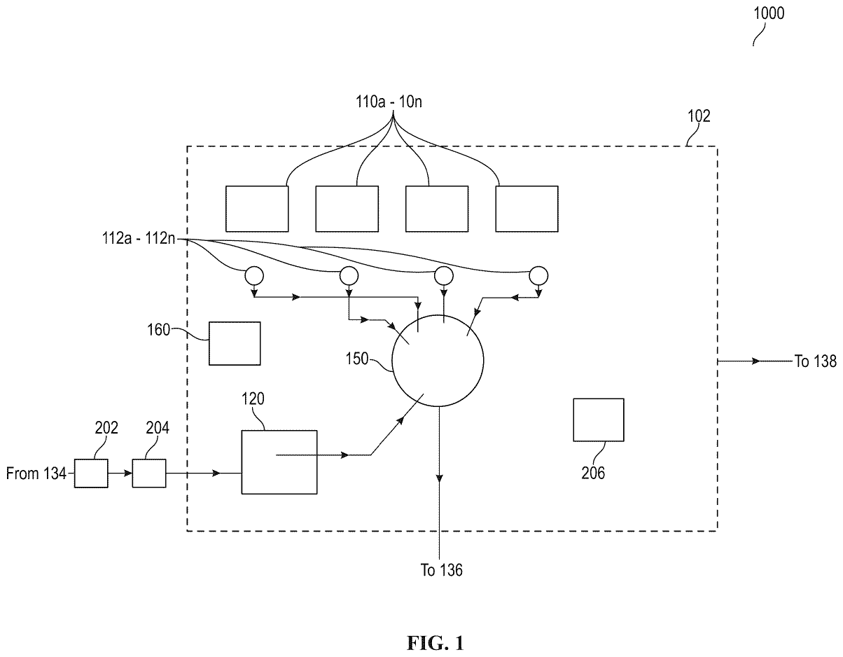

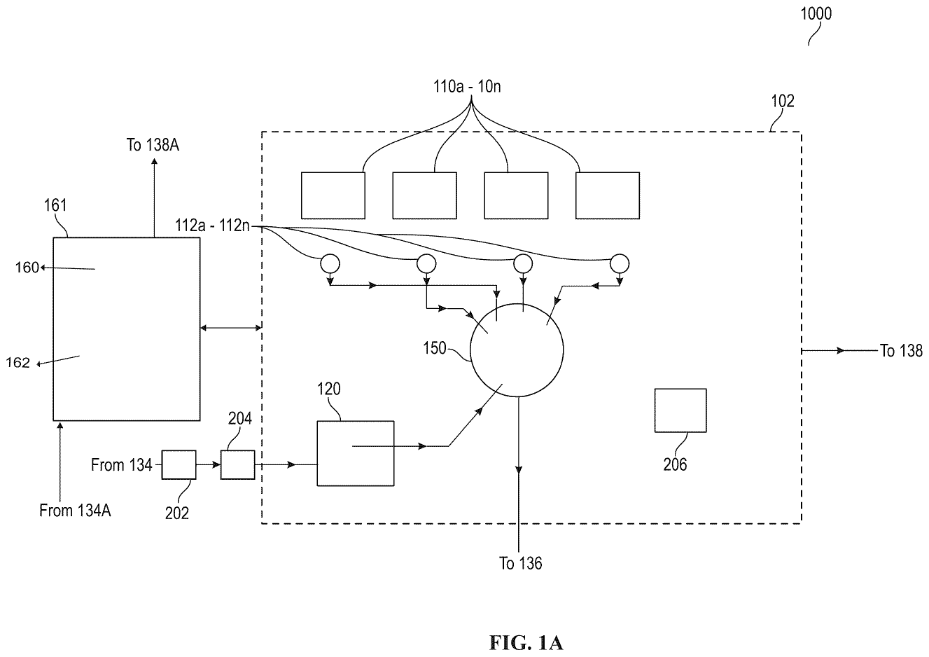

FIG. 1 is a fluidic schematic of the implantable device housing (102) illustrating the internal flow paths that interface with the multi-lumen catheter. A plurality of drug reservoirs (110a-110n) is manifolded through individually addressable microvalves (112a-112n) to a common line that feeds the pump (150). Cerebrospinal fluid (CSF) enters the housing along an inflow path labelled “From 134”, passes an inline particulate filter (202) and downstream check valve (204) that prevents CSF backflow into the drug manifold, and is directed to the CSF reservoir (120) for sampling/conditioning. Under control of the processor (170) (not shown), the pump (150) meters therapeutic agent(s) from the reservoir bank into the drug-delivery path coupled to lumen (138), while conditioned CSF and/or mixed effluent is returned via port (136) toward the catheter outflow lumen. A patient alert module (206) is coupled to the control electronics (not shown) to provide status and fault indication during refill, sampling, and closed-loop dosing operations.

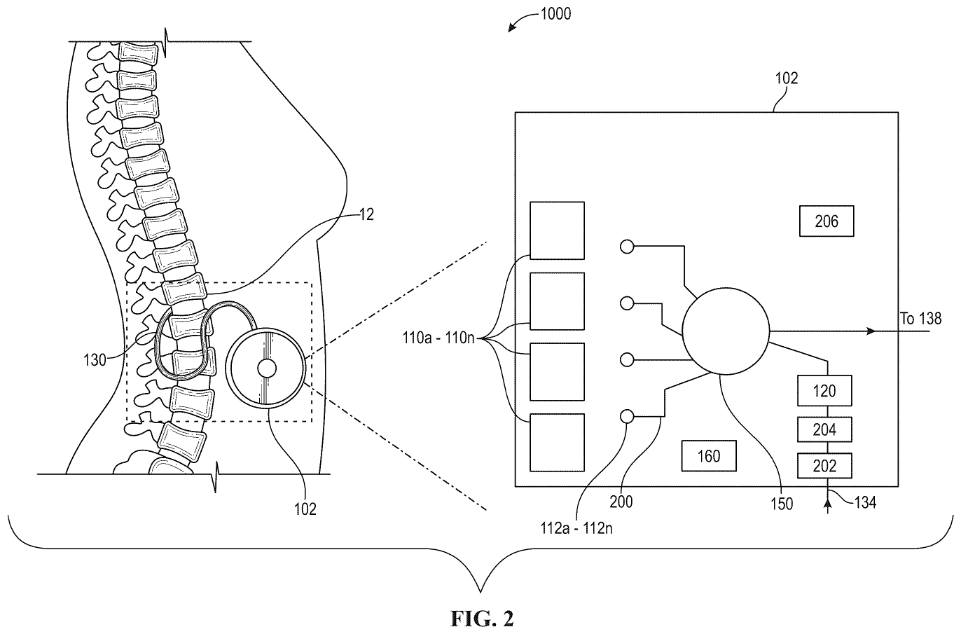

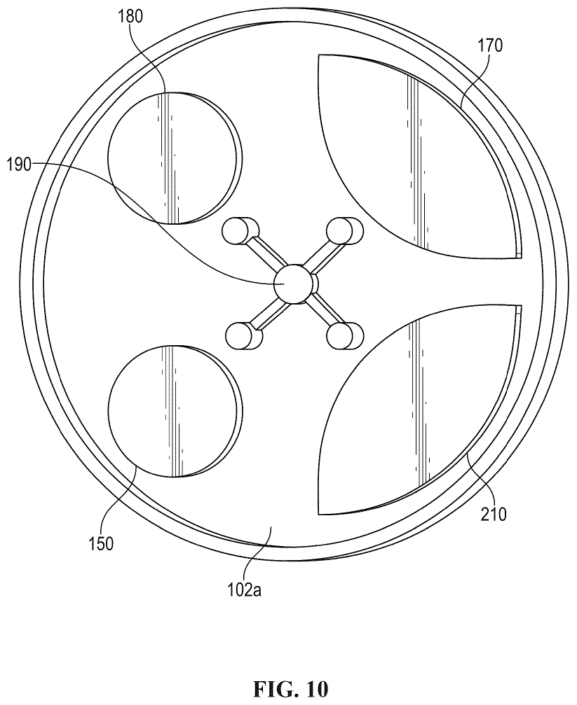

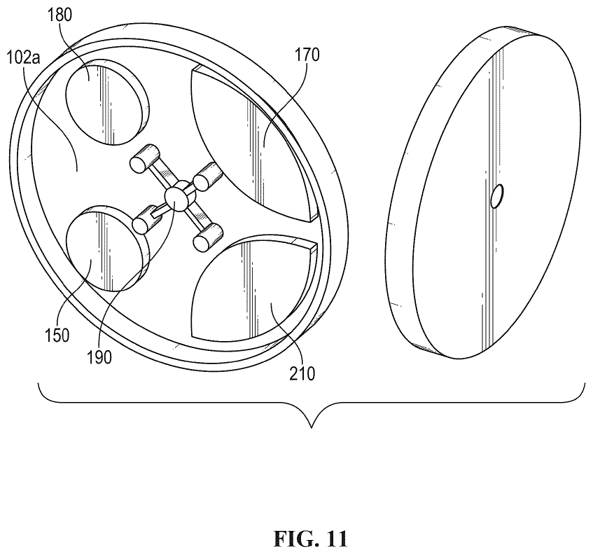

FIG. 1 and FIG. 2 illustrate the system (1000) comprising an implantable pump assembly (102) that houses reservoir modules (110a-110n) containing the therapeutic agents, a pump (150) mechanism for controlled drug infusion, and a catheter (130) configured with channels for drug delivery, cerebrospinal fluid (CSF) sampling, and biosensor (160) operation. The pump assembly (102) incorporates a processor (170) and control module that controls the dose execution and communicates wirelessly with remote clinical systems as shown in FIG. 10 and FIG. 11. In addition, the system (1000) may include refill ports and CSF sampling interfaces that facilitate reservoir (110a-110n) replenishment and sample analysis, ensuring continuous and adaptive therapeutic management. The device housing (102) has both internal component placement and external surface (102a). The housing (102) may be made up of biocompatible, non-ferromagnetic materials, the housing (102) is optimized for MRI compatibility and includes electromagnetic interference (EMI) shielding to support operation in varied clinical imaging environments. Further, the housing (102) may include smooth internal surfaces (102b) to protect delicate components such as the microprocessor and battery (210), as well as external configurations that facilitate secure fixation to the subject's anatomy. Implantation methods range from subcutaneous placement in the abdominal wall, flank areas, or submuscular pockets, depending on subject anatomy and clinical requirements. Fixation and stability mechanisms, such as suture tabs or adhesive interfaces, may be incorporated to prevent migration of the device following implantation.

In various embodiments, the intrathecal drug delivery system (1000) comprises an implantable pump housing (102), a multi-lumen catheter (130), one or more biosensors (160), a plurality of reservoirs for drugs (110a-110n) and a CSF reservoir (120), an electronic control module, a battery module (210), and wireless communication components (180). The pump (150) is configured to aspirate CSF from the intrathecal space (12), analyse for drug concentration and physiological parameters, and, based on biosensor (160) feedback, dispense precise volumes of one or more CNS-acting drugs either simultaneously or sequentially. The intrathecal drug delivery system (1000) may also be programmed to operate in a continuous flow mode, where CSF is constantly circulated through the device, or a cyclic aspiration mode, in which CSF is periodically aspirated, analysed, and reintroduced.

The intrathecal drug delivery system (1000) comprises: an implantable pump housing (102); at least one reservoir (110a-110n) adapted to contain a therapeutic agent for administration to the cerebrospinal fluid (CSF); at least one multi-lumen catheter (130) operatively coupled to the pump housing (102) and configured to establish fluid aspiration and delivery between the pump housing (102) and the intrathecal space (12) of a subject; at least one biosensor (160) configured to detect at least one physiological or chemical parameter associated with the CSF, including but not limited to drug concentration, temperature, or pressure; at least one processor (170) operatively connected to the biosensor (160) and the pump (150), the processor (170) being programmed to receive input from the biosensor (160) and to control operation of the pump (150) and/or reservoirs (110a-110n) to deliver one or more therapeutic agents to the CSF in response to biosensor (160) input; a power source (210) disposed within or operatively connected to the pump housing (102); and a user interface and/or wireless communication module (180) enabling remote programming, monitoring, and/or operation of the system (1000).

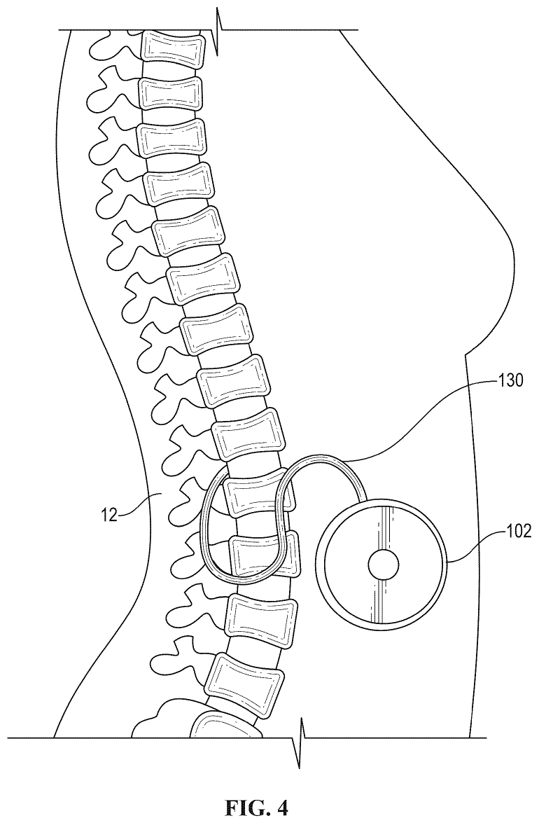

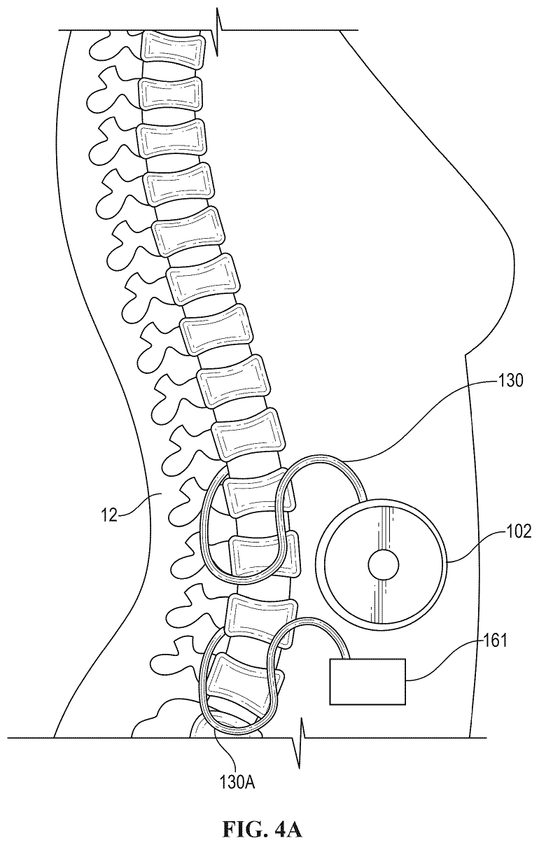

In an alternate embodiment, the biosensors (160) and drug reservoirs (110a-110n) may be integrated within an adjacent extension module (161) that is subcutaneously affixed to the pump housing (102), as depicted in FIG. 1A and FIG. 4A. The inclusion of such an extension module (161) may be determined based on the number, size, or specific configuration requirements of the biosensors (160) and/or reservoirs (110a-110n). This adjacent extension module (161) provides additional spatial capacity for accommodating these components without altering the main pump housing (102). Cerebrospinal fluid (CSF) may enter the adjacent extension module (161) through an alternate inflow pathway identified as “From 134A”, thereby enabling the biosensors (160) housed within the extension module (161) to continuously monitor various physiological parameters and measure the concentration of therapeutic agents present in the CSF. Based on sensor feedback, the pump (150) may dispense precise volumes of one or more therapeutic agents from the drug reservoirs (110a-110n) into the intrathecal space (12) via an alternate multi-lumen catheter (130A) having a drug-delivery lumen, labelled “To 138A”, thereby facilitating targeted and controlled drug delivery as illustrated in FIG. 4A.

In one embodiment, the device housing (102) is a generally disc-shaped, two-sided structure in which the external surface (102a) carries the battery (210), processor (170), wireless module (180), pump (150) and access port (190), while the internal surface (102b) carries plural drug reservoirs (110a-110n) and a CSF reservoir (120). A refill manifold hydraulically couples the access port (190) to the reservoirs via microvalves (112a-112n). The device housing (102) is made of titanium or a ceramic-titanium hybrid, featuring EMI shielding and rounded edges for atraumatic implantation. Suture tabs and an under-surface texture reduce migration.

In certain embodiments, each reservoir (110a-110n) has a nominal capacity in the range of 0.2-5.0 mL, with a refill tolerance of ±5% of the target volume, as measured by internal pressure-volume correlation during maintenance. Pediatric configurations may utilize reduced volumes consistent with maximum hourly dose limits enforced by the processor (170).

The access port (190) may feature a self-sealing septum (192) that is accessible transcutaneously, and a radiopaque or wireless locator for precise location of the access port (190) for refilling the reservoirs (110a-110n) Under sterile technique, a non-coring needle is advanced at 90° through (192) to refill (110a-110n) via the refill manifold; a separate branch provides controlled CSF sampling into (120) without interrupting therapy.

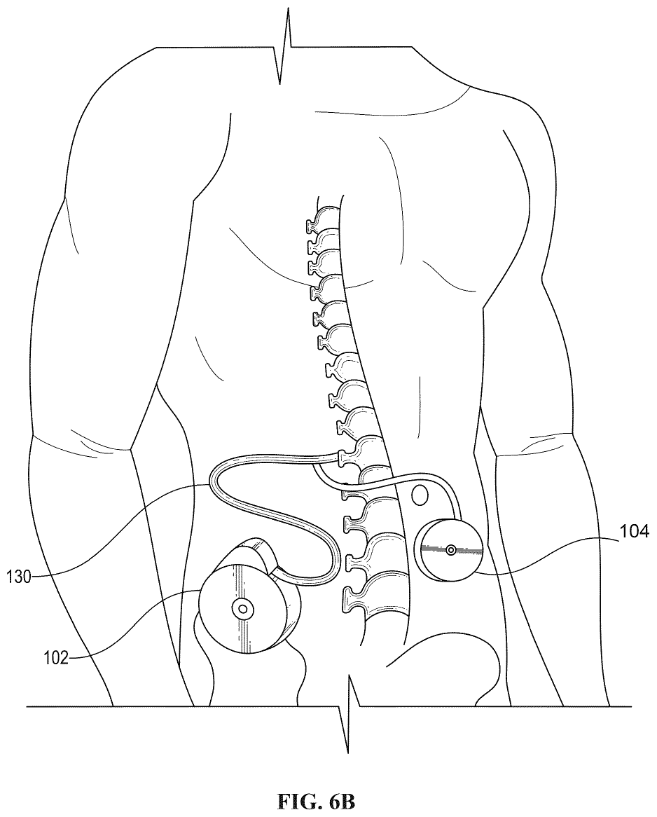

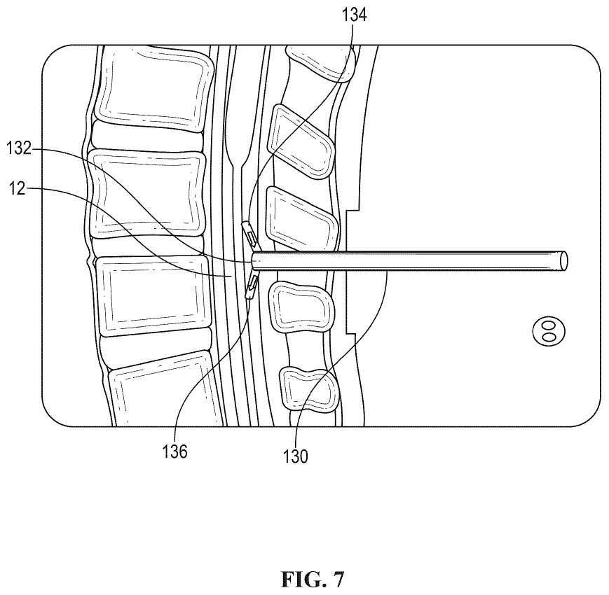

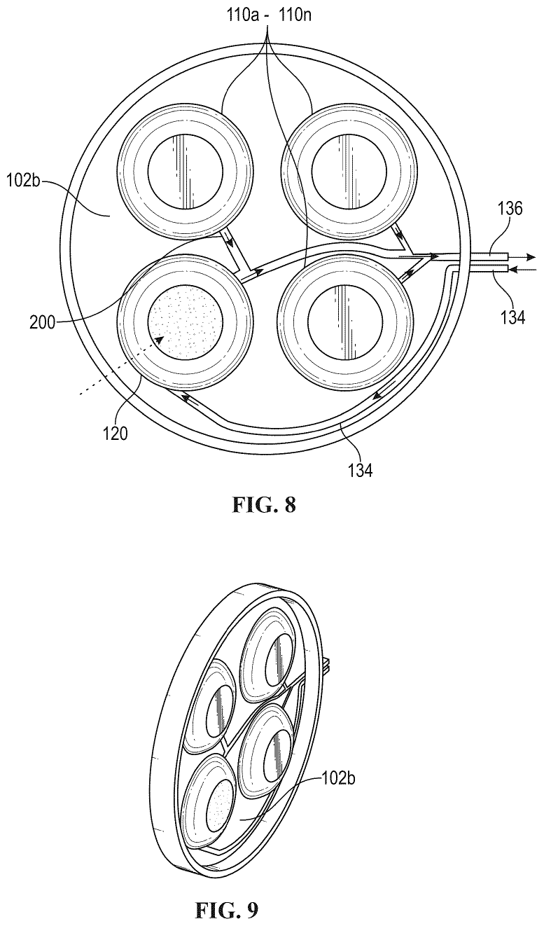

FIG. 8 shows the internal, anatomy-facing side (102b) of the implantable housing (102) with multiple drug reservoirs (110a-110n) arranged around a CSF reservoir (120). A microfluidic routing segment (200) couples the reservoirs and CSF handling paths to the catheter interface, with ports labelled 134 and 136, respectively, the CSF inflow and CSF return connections to the multi-lumen catheter. In this configuration, aspirated CSF enters via 134 for sampling/conditioning in 120 and is returned via 136. Meanwhile, drug dosing is delivered through a dedicated path (not shown in this view), thereby maintaining separation between CSF management and drug manifolding, and reducing the overall device profile and flow-path length.

FIG. 9 illustrates a variant internal-side layout (102b) emphasising the geometry of the reservoir array (110a-110n) and the microfluidic network (200) that routes toward the catheter interfaces 134 (CSF inflow) and 136 (CSF return). The CSF reservoir (120) is positioned to receive flow from 134 upstream of the drug junctions, thereby isolating CSF sampling and conditioning from the drug reservoirs. Meanwhile, the return path via 136 supports closed-loop operation. The component distribution on (102b) balances internal volume, preserves strain-relieved catheter egress, and facilitates modular servicing/replacement of fluidic modules.

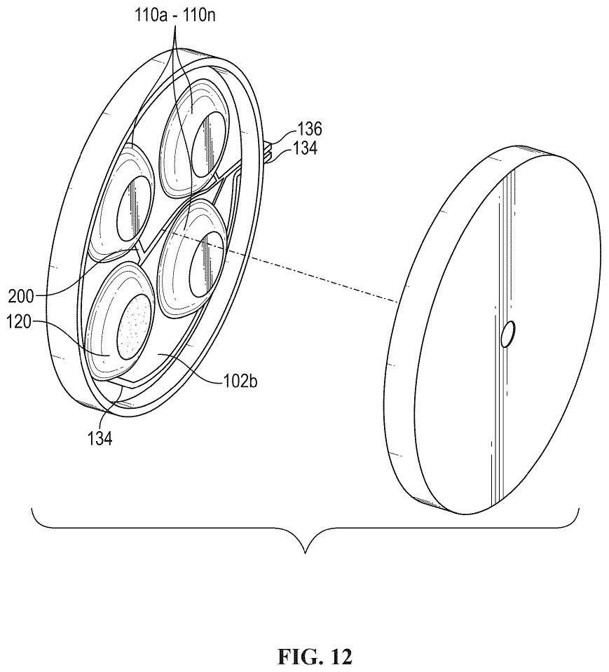

The system (1000) of the present invention, may also have a two-sided housing design (“coin” or “disc” configuration), with an external surface (102a) oriented toward the skin and an internal surface (102b) oriented toward the subject's abdomen or spinal cavity as shown in FIG. 8 to FIG. 12. The external surface (102a) typically houses the battery module (210), electronic processor/module (170), wireless antenna (for Bluetooth, Wi-Fi, NFC, or other protocols) (180), the primary pumping mechanism (150), and a central access port (190) that branches internally for refilling multiple reservoirs (110a-110n) or for CSF sampling, as shown in FIG. 10. FIG. 10 presents the skin-facing, external side (102a) of the implantable housing (102), showing the spatial arrangement of core modules that support closed-loop intrathecal delivery. In this embodiment, the battery/power source (210) and processor/controller (170) are co-located on the external side to minimise intra-pocket height while providing direct proximity to the wireless communication module/antenna (180) for reliable over-the-skin telemetry. A low-profile pump (150) is mounted on the same side and hydraulically coupled to internal reservoir manifolds (not shown in this view). A centrally accessible port (190) provides a transcutaneous entry point for sterile reservoir refill and optional CSF sampling. The illustrated layout balances service access, RF performance, and mechanical profile at the patient interface, while preserving fluidic isolation and component shielding inside the housing.

The electronic module/processor (170) can be programmed to be adaptive by the use of AI or machine learning, to learn drug delivery patterns over time for subject-specific managements. FIG. 8 illustrates the internal surface (102b) comprising multiple drug reservoirs (110a-110n), typically at least three, but possibly more, arranged in radial, parallel, or stacked configurations, as well as a CSF reservoir (120) and associated biosensors (160), including a near-infrared (NIR) biosensor for measuring drug concentration and a temperature biosensor for monitoring CSF temperature. All housing materials are selected for their biocompatibility and hermeticity, including, but not limited to, medical-grade plastics, ceramics, stainless steel, titanium, and tantalum. The housing (102) may be further shielded to prevent electromagnetic interference (EMI) or radiofrequency interference from nearby medical equipment.

FIG. 11 shows the skin-facing, external side (102a) of the implantable housing (102), illustrating the placement of the processor/controller (170) and battery/power source (210) in proximity to the wireless communication module/antenna (180) to support reliable over-skin telemetry, with a low-profile pump (150) mounted on the same side and a centrally accessible access port (190) for transcutaneous reservoir refill and optional CSF sampling. This external-side layout provides service access at the patient interface while preserving internal fluidic isolation and a compact profile suitable for subcutaneous implantation.

In certain embodiments, system's reservoirs, includes one or more therapeutic agent reservoirs (110a-110n). These reservoirs (110a-110n) may be detachable for ease of refill or replacement, and may also have a dedicated CSF reservoir(s) (120) that allow for intermittent sampling and analysis, thereby ensuring that both drug therapy and CSF homeostasis can be continuously monitored. Controlled release mechanisms are implemented via microvalves (112a-112n) that regulate the dispensing of the drug formulation from the reservoirs (110a-110n).

Internal, anatomy-facing side (102b) of the implantable housing (102) showing an array of plural drug reservoirs (110a-110n) arranged around a CSF reservoir (120) and interconnected by a microfluidic routing segment (200) as illustrated in FIG. 11. The housing interfaces with the multi-lumen catheter via discrete ports labelled 134 (CSF inflow) and 136 (CSF return), enabling aspirated CSF to enter through 134, be directed to 120 for sampling/conditioning upstream of any drug junctions, and be returned to the intrathecal space via 136. In this embodiment, drug dosing is routed on a dedicated path (not shown here) to maintain fluid separation between CSF handling and the drug manifolding, thereby supporting closed-loop operation and minimising cross-contamination.

In one embodiment, the catheter (130) includes at least four discrete lumens: (134) for CSF inflow, (136) for CSF outflow, (138) for drug delivery, and (140) for the power/data lumen. The power/data lumen (140) carries insulated conductors for powering an optical emitter/photodetector pair, as well as return conductors for analogue and digital signals. In a variant, (140a) carries power and (140b) carries bidirectional data to reduce crosstalk.

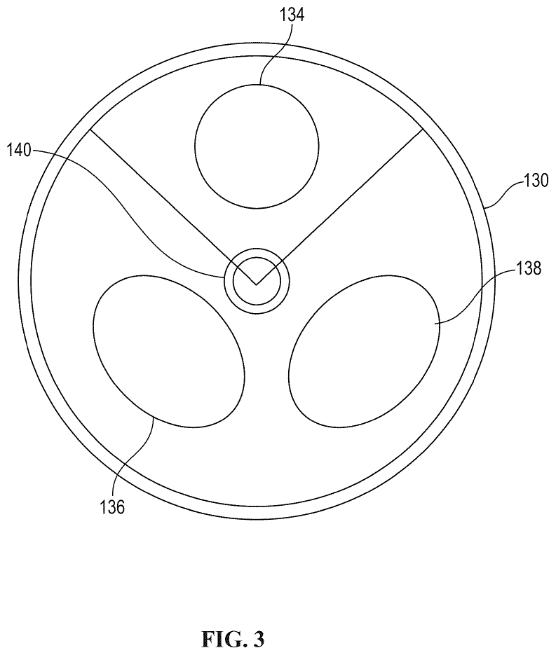

In certain embodiments, the valving system is integrated with the pump mechanism (150) and the processor's dosing, ensuring that drug delivery is adjusted in real time based on feedback from the biosensors (160). The multi-lumen catheter (130) has multiple independent channels, each built to ensure the simultaneous management of several functions. One or more lumens are tasked exclusively with the infusion of the drug formulation into the intrathecal space (12), while additional lumens may be dedicated to managing CSF inflow (134) and outflow (136) for sampling purposes as shown in FIG. 3.

FIG. 3 shows a transverse cross-section of the multi-lumen catheter (130) configured to provide discrete pathways for cerebrospinal fluid (CSF) management, drug infusion, and sensor support. As illustrated, the catheter body defines separate lumens including a CSF inflow lumen (134), a CSF outflow lumen (136), a drug-delivery lumen (138), and a power/data lumen (140) for power and/or data transmission to an intrathecal biosensor. The lumens are fluid-isolated by internal septa to minimise cross-communication. They are dimensioned to maintain physiologic aspiration/return rates while preserving flexibility for atraumatic placement along the spinal canal. This enables simultaneous CSF sampling and return, as well as targeted intrathecal infusion, while accommodating the conductors or optical elements required for closed-loop sensing.

In certain embodiments, a separate lumen (140) is provided to power the integrated near-infrared (NIR) biosensor (160) and to transmit sensor data back to the central controller (170). The catheter (130) ensures optimal biosensor integration; in some variations, the biosensor (160) is located at the distal tip (132) to directly sample the CSF and measure drug concentrations, whereas in other configurations the biosensor (160) may be integrated within the device housing (102) with a conduit extending to the catheter tip (132).

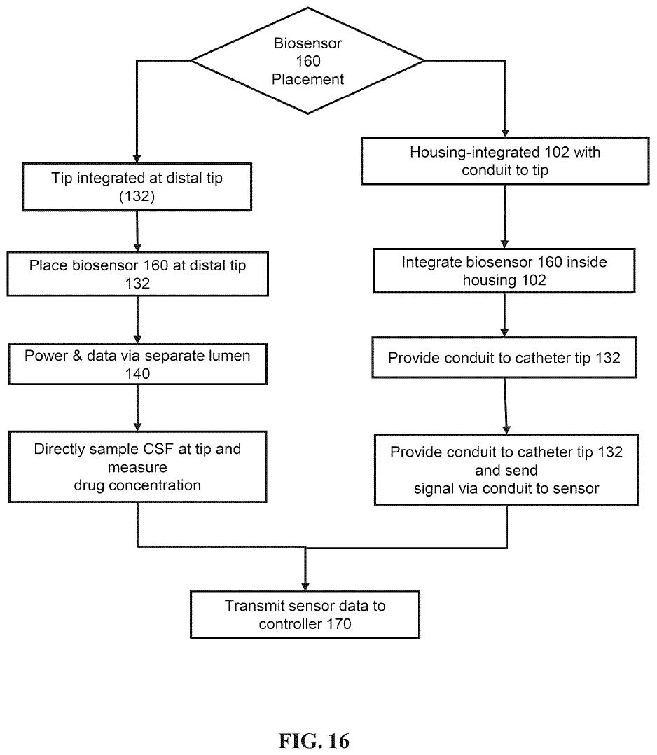

In another embodiment, the intrathecal system (1000) is implanted with the device housing (102) in a subcutaneous pocket and the multi-lumen catheter (130) routed to place its distal tip (132) in the intrathecal space (12). The clinician then executes the “Biosensor 160 Placement” routine as shown in FIG. 16, selecting between two sensor architectures. In the first, the biosensor (160) is integrated at the distal tip (132) so that the sensor directly interrogates CSF at the point of delivery; electrical conductors for power and data are routed through a dedicated power/data lumen (140) within the catheter body to the controller (170) in the housing. During operation, the tip-mounted sensor emits/receives signals in situ, computes or relays raw measurements indicative of drug concentration, and transmits sensor data to the controller (170) for downstream processing, logging, and (in closed-loop embodiments) infusion control. In the alternative, the biosensor (160) is integrated inside the housing (102). A conduit to the catheter tip (132), implemented within the same lumen (140), provides an optical and/or electrical path, allowing the CSF at the tip to be interrogated through the conduit. At the same time, the sensing elements remain in the housing. In this housing-integrated variant, the signal generated at the tip window is returned via the conduit to the biosensor in (102), and derived concentration data are forwarded to the controller (170). In both branches, the system preserves a segregated signal pathway in the lumen (140) that is independent of drug-delivery and CSF-handling lumens, thereby maintaining fluidic isolation while enabling reliable, real-time monitoring at the anatomical site of interest.

In certain embodiments, a temperature sensor may be disposed within or adjacent to the CSF reservoir (120) for continuously monitoring of CSF temperature. Further, the temperature sensor detects physiological conditions indicative of elevated body temperature, such as fever or an inflammatory response. Upon detection of a temperature exceeding a predetermined threshold indicative of fever or inflammatory response, the processor is programmed to inhibit the pump (150) or modulate the dosage accordingly. The drug administration is dynamically adjusted based on real-time physiological parameters, thereby enhancing subject safety and therapeutic efficacy.

In certain embodiments, the catheter (130) is fabricated from biocompatible, non-ferromagnetic materials that ensure safe long-term implantation while maintaining the mechanical flexibility needed for accurate drug dispersion and CSF sampling. A central component of the system's (1000) monitoring capability is the biosensor (160) based on NIR spectroscopy. This sensor (160) comprises one or more laser diodes that emit near-infrared light and photo detectors that capture the resulting back-scattered or transmitted light from the CSF. The device employs robust spectroscopic analysis methodologies, whereby the optical absorption data captured are correlated to specific drug concentrations through calibration curves and validated analytical models.

FIG. 4 illustrates the implantable intrathecal drug-delivery system positioned in situ, with the device housing (102) placed subcutaneously and a multi-lumen catheter (130) tunneled toward the spine to access the intrathecal space (12). The view depicts the anatomical relationship between the subcutaneous housing and the routed catheter, which follows a gentle strain-relief as it exits the housing and traverses soft tissue before entering the intrathecal region for targeted delivery and CSF handling. In this embodiment, distinct catheter flow paths (e.g., for CSF inflow/return and drug infusion) are provided within the catheter body (not individually labelled in this view) to enable simultaneous sampling and dosing. The subcutaneous placement facilitates stable fixation and percutaneous access to the housing for refill and service.

In another embodiment as illustrated in FIG. 4A, an adjacent extension module (161) is affixed to or closely apposed with the device housing (102) within the same subcutaneous pocket. The extension module (161) houses at least one of: (i) additional drug reservoirs (110a-110n); (ii) at least one biosensor (160); and/or (iii) microfluidic routing (200) that interfaces with the catheter (130A). Electrical and/or optical connectivity between (161) and (102) is provided through the power/data lumen (140) and a short intra-pocket harness, while fluidic paths couple to the drug-delivery lumen (138A) and CSF lumens (134A) via strain-relieved connectors. In a tip-sensor configuration, (161) may carry the NIR sensor electronics and drivers, with only the emitter/detector head located at the distal tip of the catheter (130A). During operation, the tip-mounted biosensor (160) actively emits and receives optical or electrical signals in situ to assess concentrations of therapeutic agents directly within the CSF. The biosensor (160) may compute measurements locally or relay raw signal data to the controller (170), which performs signal processing, data logging, and, in closed-loop configurations, uses the processed data to dynamically adjust infusion rates in real time.

In an alternative housing-sensor configuration, the biosensor (160) remains within the extension module (161), but operates via an optical conduit routed through the power/data lumen (140). This conduit terminates at a clear optical window located near or at the tip of the catheter (130A). Light signals (e.g., excitation and emission wavelengths) are transmitted and received through the conduit, allowing localized sensing while maintaining bulkier sensor components within the adjacent extension module (161). The biosensor (160) output is then transmitted to the controller (170) wirelessly or through the intra-pocket harness for further processing, logging, and therapeutic control. The (161) variant reduces the footprint of (102) while preserving closed-loop sensing and dosing.

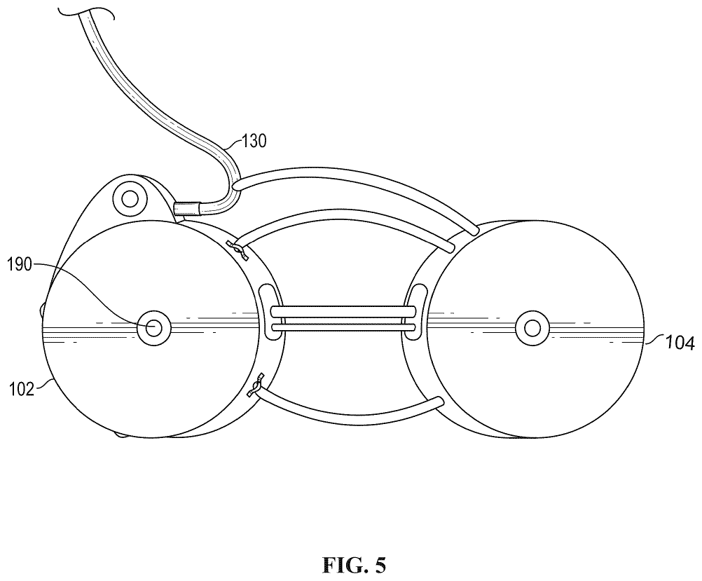

FIG. 5 illustrates the implantable device housing (102) with an integrated access port (190) and the proximal interface to the multi-lumen catheter (130). The access port (190) is positioned on the skin-facing side of the housing to permit transcutaneous needle entry for reservoir refill and optional CSF sampling. At the same time, the catheter (130) exits the housing through a strain-relieved outlet, allowing it to be routed toward the intrathecal target site. The view illustrates the relative positioning of the port and catheter opening on the housing, indicating the fluidic and mechanical interfaces by which the internal reservoir/manifold set communicates with the catheter's dedicated lumens (not individually depicted in this figure). The arrangement supports aseptic servicing without explanation and maintains separation between infusion and CSF pathways for closed-loop operation.

In one embodiment, the device housing (102) and the catheter (130) may be detachably connected to an auxiliary housing (104) implanted subcutaneously through the use of suture tabs as shown in FIG. 5. The auxiliary housing (104) may be configured to accommodate various components, including biosensors (160), drug reservoirs (110a-110n), electronic control module (170), battery module (210), and wireless communication module (180). The device housing (102) and the auxiliary housing (104) may be positioned on opposite sides of the patient's abdomen, while being operatively linked via a common multi-lumen catheter (130) that enables coordinated delivery and monitoring functions. Further, the auxiliary housing (104) may be independently controlled via the wireless communication module (180).

With reference to FIG. 5, when present, the extension module (161) is fluidically integrated with the refill manifold, addressed by the access port (190) and self-sealing septum (192) on the housing (102), thereby allowing the refill of reservoirs resident in (161) without requiring a second port. In other embodiments, (161) includes a dedicated service port (190a) (structurally similar to 190) to permit the selective refill or purge of (161) reservoirs or CSF channels. Both ports are keyed to prevent misconnections, and the processor (170) enforces maintenance interlocks, ensuring that only the intended vessel is accessed during refilling or sampling.

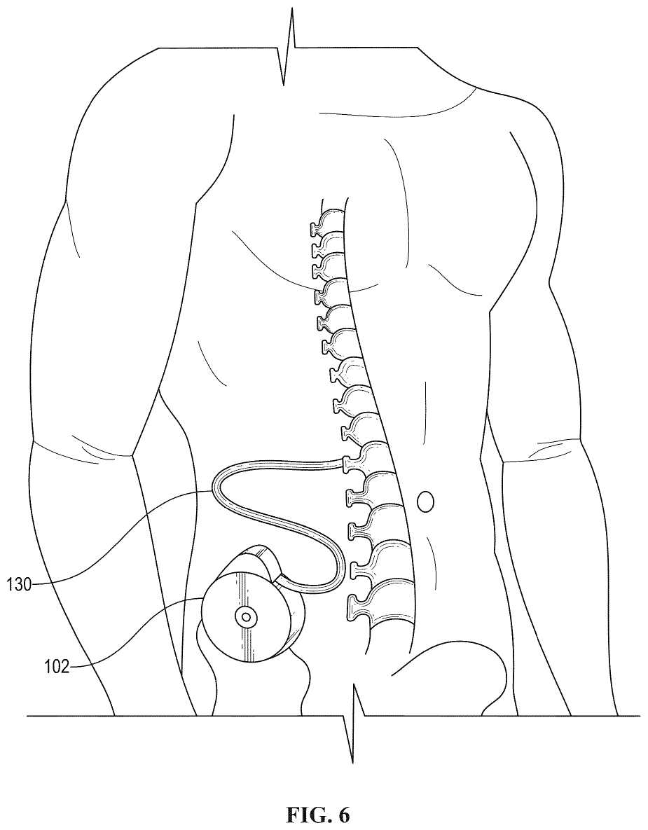

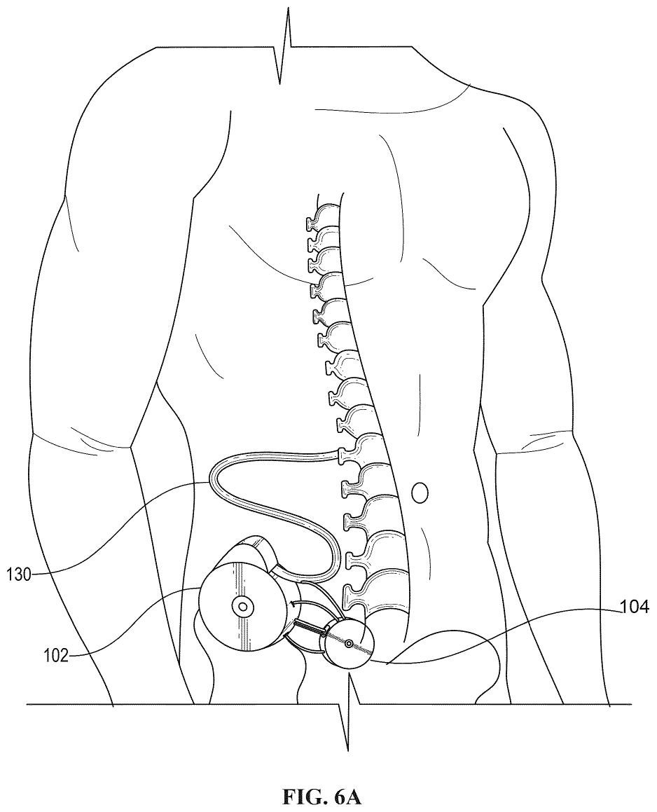

An implanted assembly (in FIGS. 6a and 6b) shows the subcutaneous orientation of the device housing (102) and routing of the multi-lumen catheter (130) from the housing exit toward the spinal access site. The view emphasises a gentle, strain-relieved catheter path with a low-profile subcutaneous tunnel to reduce kinking, migration, and local tissue stress, while maintaining sufficient bend radius for long-term durability. In certain embodiments, proximal fixation features adjacent the housing help stabilize the catheter as it transitions from the pocket to deeper tissues, and the routing preserves the internal separation of the catheter's flow paths (e.g., drug infusion, CSF inflow/return, and sensor leads, not individually labeled in this figure) to support simultaneous dosing and sampling as part of a closed-loop.

As shown in FIG. 6A, the extension module (161) occupies a low-profile recess adjacent to housing (102) and is secured by a perimeter bracket and/or suture tabs. A short, flexible interface couples (161) to (102): (i) a fluidic umbilical with keyed connectors to the drug-delivery path (138) and CSF paths (134/136); and (ii) a signal umbilical that links (161) to the processor (170) and wireless module (180) through sealed feedthroughs. This arrangement enables modular scaling e.g., adding reservoirs or sensor capacity in (161) without increasing the vertical profile of (102) under the skin.

In another configuration of FIG. 6B, the auxiliary housing (104) may provide bulk drug storage and battery (210), while the extension module (161) remains adjacent to (102) to host high-speed sensor electronics (160) and/or microfluidic routing (200) local to the controller (170). Catheter (130) routes between (102) and the intrathecal space (12), and inter-housing conduits couple (104) to (102) for reservoir feed. The processor (170) arbitrates sources (e.g., from 104 vs. 161) and enforces dose and safety limits during synchronized operation via wireless (180).

In one embodiment, the auxiliary housing (104) may be affixed to and positioned adjacent to the device housing (102) on the same side of the patient's abdomen, as illustrated in FIG. 6a. This configuration allows for a compact and integrated arrangement of the device components within a localized implantation site. In an alternate embodiment, the auxiliary housing (104) and the device housing (102) may be implanted on opposite sides of the patient's abdomen, as depicted in FIG. 6b. This spatial separation facilitates distribution of the device components across the abdominal region, which may be advantageous for accommodating larger or additional modules, optimizing patient comfort, or improving surgical access and implantation flexibility.

A dual-lumen intrathecal catheter (130) as illustrated in FIG. 7 having discrete passageways for CSF inflow (134) and CSF return (136) that extend to a distal tip (132) positioned within the intrathecal space (12). The parallel lumens maintain fluid isolation along the catheter body, allowing CSF to be aspirated via lumen (134) into an implant housing for analysis or conditioning, and then directed back toward the intrathecal space via lumen (136), optionally with a metered therapeutic agent entrained in the return flow. This arrangement enables closed-loop sampling and delivery while preserving atraumatic placement and flexibility for subcutaneous routing.

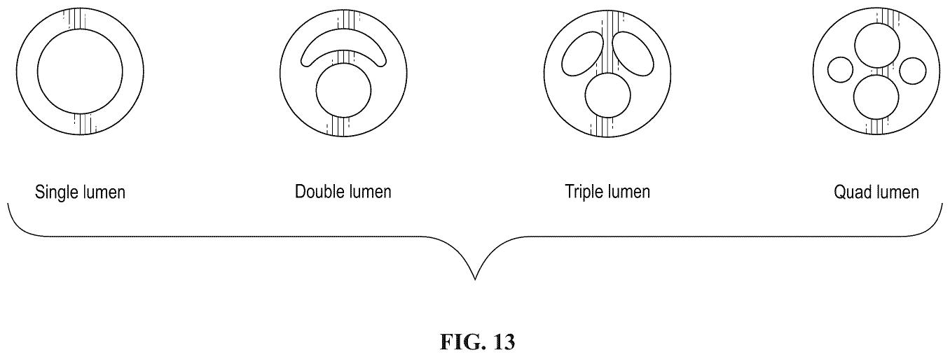

The multi-lumen catheter (130) connecting the device housing (102) to the intrathecal space (12) may have two, three, four, or more lumens as shown in FIG. 13. A typical dual-lumen configuration comprises one lumen for CSF inflow (134) and another for CSF and drug outflow (136) as shown in FIG. 7; alternative embodiments feature additional lumens for independent drug delivery (138), in-line sensors, or for the introduction of nanomedicines or nanobots, as depicted in FIG. 3. The catheter (130) may be secured and anchored along the spinal column or at specific vertebral levels using fixation devices, with its shape and flexibility tailored for optimal fit in pediatric, adult, or veterinary use, as depicted in FIG. 4, FIG. 6a and FIG. 6b. The distal tip (132) and ports of the catheter (130) can be straight, curved, side-fenestrated, or end-facing, and may include radioopaque markers or antimicrobial coatings. Catheter (130) materials may include biocompatible plastics, silicone, or elastomers, and the cross-sectional geometry of the lumens may be circular, oval, or irregular.

A central electronic control module, comprising a processor (170) and associated electronics, coordinates the system's operation. The processor (170) receives continuous or periodic input from biosensors (160), including drug concentration, CSF temperature, pressure, pH, osmolarity, or other physiological or environmental parameters (e.g., allergens, ambient temperature), and uses these inputs to dynamically adjust drug dosing via artificial intelligence or machine learning algorithms. The machine learning algorithm includes, but is not limited to, supervised learning, unsupervised learning, and reinforcement learning. In another embodiment, the machine learning algorithm includes decision trees, random forest, support vector regression, hierarchical clustering, deep Q-network, and the like. The artificial intelligence algorithm includes, but is not limited to, recurrent neural networks (RNNs), convolutional neural networks (CNNs), generative adversarial networks (GANs), and long short-term memory networks (LSTMs). Safety features are embedded within the processor (170) programming, including safeguards against overdosing, real-time alerts for reservoir (110a-110n) depletion, flow obstruction, sensor failure, or device tampering, and a programmable “lockdown” mode to prevent unauthorized use.

The biosensor (160) may include, but not limited to, near-infrared sensors for drug quantification, temperature sensors, environmental sensors, pressure transducers, and biomarker detection modules (e.g., for proteins, inflammation, infection). Biosensors (160) can be integrated within the housing (102), embedded in the catheter (130), or even implemented as separate modules connected via wired or wireless communication. For advanced applications, microfluidic channels (200) and nanoscale features may support the delivery of nanobots, nanoparticles, viral vectors, gene therapies, or antibody therapies. Each microfluidic channel (200) may include inline particulate filters (202) that remove particulate contaminants or drug precipitates, and prevent catheter blockage. Further, check valves (204) may be located downstream from the inline particulate filters (202) to maintain unidirectional drug delivery. The check valve (204) is a passive microfabricated component with one way flow functionality that prevents back flow of the CSF, thereby protecting drug reservoirs (110a-110n) from contamination or dilution.

Downstream of each reservoir (110a-110n), a microfluidic channel (200) incorporates an inline particulate filter (202) (e.g., 2-5 μm pore) to capture particulates or precipitates, followed by a passive check valve (204) to prevent CSF backflow. The path merges into the drug-delivery lumen (138) proximal to the catheter strain-relief.

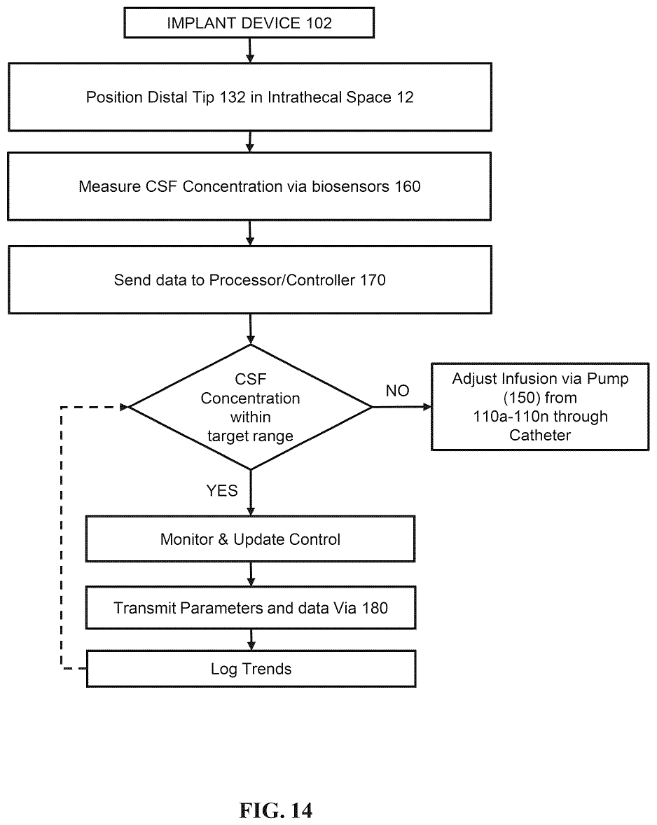

In an embodiment, the implantable device (102) is placed in a subcutaneous pocket and secured using standard surgical techniques, thereby initiating the therapy routine shown in FIG. 14. The distal tip (132) end of the multi-lumen catheter is then advanced to the intrathecal space (12) and fixed at the target vertebral level (flowchart step “Position Distal Tip 132 in Intrathecal Space 12”). At programmed intervals (e.g., every 60-300 s), the intrathecal biosensor (160) acquires a measurement of CSF drug concentration (“Measure CSF Concentration via biosensors 160”) and forwards raw or preprocessed signal data to the on-board processor/controller (170) (“Send data to processor/controller 170”). The controller evaluates whether the concentration lies within a preset therapeutic window (decision block “CSF Concentration within target range—Yes/No”). If Yes, the controller maintains the current set-point and execution state (“Monitor & Update Control”), transmits the current parameters and the most recent measurements for clinical review (“Transmit parameters and data via 170”), and appends the values to a rolling log (“Log Trends”). If No, the controller computes a corrective action and adjusts infusion via the pump (140) by metering the therapeutic agent from one of the reservoirs (110a-110n) through the catheter (“Adjust Infusion via Pump (150) from 110a-110n through Catheter”), then re-samples after a guard interval to verify return to range before resuming monitoring, telemetry, and trend logging.

In one embodiment, an initial baseline infusion is set (e.g., 0.05-0.20 mL/h), and out-of-range corrections are applied as bounded micro-boluses (e.g., ≤25 μL each) with a minimum inter-bolus interval (e.g., ≥60 s) to avoid overshoot. Reservoir selection (e.g., 110a for Drug A versus 110b for Drug B) is determined by the active therapeutic program. Each actuation event is logged with a timestamp and associated sensor value in the controller's nonvolatile memory, and these records are packaged in the outbound telemetry frame for clinician dashboards. When the concentration returns to the target band on two consecutive samples, the controller returns to steady-state monitoring with adaptive trimming of the baseline rate; if the value remains out of range beyond a maximum correction count, the system transitions to a safe state (infusion hold, alert issuance) pending clinician input.

In another embodiment, an NIR biosensor (160) is integrated at the catheter distal tip (132) and optically interrogates CSF in situ. The emitter produces pulsed light between 700 and 1000 nm; the detector measures backscatter/transmission. The processor (170) executes a calibration transform (e.g., PLS on baseline-corrected spectra) to estimate the drug concentration. If the result falls outside the range [Cₘᵢₙ, Cₘₐₓ], the controller actuates (112a-112n) and (150) to increase, suspend, or taper the infusion via the lumen (138). This reduces fluid handling relative to aspirate/return workflows.

In certain embodiments, the system (1000) may include miniature sensors embedded at the tip (132) of the multi-lumen catheter (130) rather than within the pump housing (102), allowing direct contact with the cerebrospinal fluid (CSF) in the intrathecal space (12). The system (1000) may utilize near-infrared (NIR) technology, employing laser diodes that emit light into the spinal fluid, capitalizing on the differential absorption properties of different drugs to assess their concentration within CSF. These in-line sensors may monitor critical parameters, such as CSF temperature, pressure, and biomarker levels, in real time. The collected data is then transmitted to a processor (170), enabling precise dose adjustments. Thus, CSF monitoring may be performed intrathecally at predetermined sampling frequency, with the required drug dose being dispensed from the reservoir (110a-110n) into the intrathecal space (12) as needed.

The wireless communication module (180) enables real-time data transmission and device programming via secure, encrypted protocols, supporting connection to mobile applications, hospital systems, or cloud-based provider dashboards. Biometric authentication, RFID, or multi-factor security protocols protect device access and control. The system (1000) may issue haptic, audible, or visual alerts to the subject or transmit notifications to healthcare providers, particularly in the event of a critical system status or maintenance requirement.

In an embodiment, the system (1000) may use one or more NIR laser diode-based biosensor (160) that emit light in a wavelength range typically between 700 nm and 1000 nm chosen for their ability to penetrate biological tissues and for their sensitivity to the unique absorption characteristics of many therapeutic molecules. When the NIR light is emitted into the cerebrospinal fluid, the light interacts with the molecules of the administered drug. Depending on the concentration and chemical properties of the drug, a portion of the light is absorbed while the remainder is scattered or transmitted. Photodetectors within the biosensor (160) are arranged to capture this back-scattered or transmitted light. The sensor (160) circuitry then converts the variations in detected light intensity into an electrical signal that reflects the drug's concentration in the fluid.

In one implementation, near-infrared spectra (700-1000 nm) are acquired at a resolution of 2-10 nm through a tip-mounted window adjacent to the intrathecal flow. Spectra are baseline-corrected and normalised before application of a multivariate calibration (e.g., partial least squares, PLS) to estimate drug concentration. The calibration is generated from subject-matched CSF matrices spanning the intended therapeutic range and validated with k-fold cross-validation. An error metric (e.g., RMSECV) and limit of detection (LoD) are stored with the model and checked at runtime.

By way of example, calibrations were obtained for: (i) morphine sulfate 0.01-1.0 mg/L (core wavelengths ~760-820 nm; RMSECV ≤0.05 mg/L); (ii) baclofen 0.005-0.5 mg/L (features~720-780 nm; RMSECV ≤0.02 mg/L); and (iii) methotrexate 0.01-2.0 mg/L (features ~830-900 nm; RMSECV ≤0.06 mg/L). Interference studies with protein (albumin 20-50 mg/dL) and variable hematocrit showed ≤10% bias after a two-point in-situ update using freshly sampled CSF via reservoir (120).

The processor (170) performs periodic model-verification using (i) single-point check standards introduced during maintenance; or (ii) opportunistic CSF samples compared against a reference cuvette in the housing-integrated variant. If model quality metrics exceed stored bounds, the controller triggers a recalibration routine or reverts to a conservative open-loop fallback until validation is restored.

In certain embodiments, the system (1000) may comprise of pump (150) and fluid control mechanism, which may utilize various technologies, including peristaltic, osmotic, or diaphragm-based pumps. The pump (150) may be integrated within the implantable device housing (102) and operates in close coordination with the processor (170). A dedicated power supply (210), typically involving an integrated battery with provisions for recharging or replacement, supports uninterrupted operation of the system (1000). The pump (150) mechanism is characterized by control methods that involve processor (170) driven infusion machine learning algorithms based on input from the biosensors (160) and additional system monitors; this allows fine-tuning of the drug delivery rate in real time to adapt to changing physiological needs.

A high-performance microprocessor may also be employed, running firmware that supports closed-loop feedback control algorithms. These algorithms integrate subject-specific pharmacokinetic and pharmacodynamic models to analyze the sensor (160) data captured from the CSF and determine the optimal dosing regimen. The processor (170) is further tasked with coordinating the operation of the pump (150), managing data from additional sensors (162), and maintaining communication with remote clinical systems. In some embodiments, the device may offer remote therapeutic management, allowing clinicians to adjust treatment parameters remotely based on real-time data analytics and secure system status reports.

Wireless communication (180) is seamlessly integrated into the system (1000) to enable remote monitoring and control. The wireless connectivity module (180) supports standard communication protocols such as Bluetooth, radio frequency (RF), or even proprietary secure systems with robust encryption and multi-factor authentication protocols to safeguard data integrity and subject privacy. The system (1000) offers remote access capabilities through various interfaces including clinical workstations, mobile applications, or web-based monitoring platforms, ensuring that system operation and dosing parameters can be adjusted securely and efficiently from remote locations. User and clinician interaction with the system (1000) is facilitated by a multi-modal interface that may include alert modules (206) offering auditory, visual, or haptic feedback. Interface screens provide real-time system notifications, dosing histories, and diagnostic data. Furthermore, the device incorporates authentication methods to ensure that only authorized personnel can access and modify the system's operational parameters, thereby enhancing overall safety. The system (1000) is also adaptable for specialized therapeutic applications. For pediatric, geriatric, and even veterinary uses, modifications in reservoir (110a-110n) size, dosing algorithms, and catheter (130) dimensions may be introduced to suit specific subject populations. The system (1000) is compatible with advanced therapies such as gene therapy, nanoparticle-based drugs, antibody therapies, viral vector administration, and even emerging technologies like nanobot delivery.

The wireless communication module (180) comprises at least a radio transceiver and antenna supporting one or more protocols (e.g., BLE, Wi-Fi, NFC) with session-level encryption and rotating keys. Access control supports biometric authentication, RFID challenge-response, or cryptographic tokens (multi-factor). Telemetry frames include timestamps, dosing history, battery status (210), and integrity checks; inbound programming updates are validated prior to application.

The device is compatible with all common medical imaging technologies including MRI, ultrasound, CT, and X-ray using non-ferrous, non-magnetic, and radio-opaque materials. The device and catheter (130), including the refilling port (190) may be equipped with radiographic markers or features that facilitate accurate localization and monitoring post-implantation.

In further embodiments, the system (1000) may be integrated as part of a broader therapy management platform, with the capability to synchronize delivery with external devices such as deep brain stimulators, spinal cord stimulators, dialysis machines, or physiotherapy equipment. The dosing can be programmed to deliver increased medication during therapy sessions and reduce dosages at rest, with real-time adjustments based on physiological monitoring and feedback.

The system (1000) allows for flexibility, modularity, and future-proofing. All core components, including pumps (150), reservoirs (110a-110n), sensors (160), electronic modules, wireless modules (180), and catheters (130), may be configured as modular, upgradable, and replaceable units, facilitating hardware or software updates and adaptation to evolving clinical or technological requirements.

In another embodiment, the present invention provides a method for delivering a therapeutic agent into an intrathecal space (12) of a subject using an intrathecal drug delivery system (1000), the method comprising the steps of aspirating cerebrospinal fluid (CSF) from the intrathecal space (12) into a CSF reservoir (120) within a pump housing (102) through a multi-lumen catheter (130); monitoring one or more parameters associated with the CSF using at least one biosensor (160); receiving and processing input received from the biosensor (160) using a processor (170) and controlling the actuation of at least one reservoir valve (112a-112n) to dispense a therapeutic agent based on the biosensor (160) feedback; and delivering precise volumes of one or more therapeutic agents sequentially into the intrathecal space (12) through the multi-lumen catheter (130).

In one example, the system (1000) is configured for adaptive dosing of analgesics wherein the biosensor (160) continuously monitors CSF drug concentrations and the processor (170) modulates the pump's output to ensure optimal pain management. In another example, the modular reservoir design allows for facile reservoir replacement and secure, sterile refill procedures, thereby minimizing downtime during chronic therapy. In yet another example, system (1000) may be used in a remote clinical management of intrathecal chemotherapy, where clinicians remotely adjust dosing based on real-time feedback from the device, significantly enhancing subject safety in oncology applications. Also, an integrated system may work in tandem with additional implantable neurological devices to provide synchronized therapy for complex neurological disorders, ensuring that drug delivery correlates seamlessly with neurostimulatory interventions. The intrathecal drug delivery system (1000) may be suitable for long-term use in chronic conditions and neurological and psychiatric disorders that require meticulous management of medication levels.

In another embodiment of the present invention, the integrated near-infrared biosensor (160) is positioned at the distal tip (132) of the multi-lumen catheter (130) such that it resides directly within the intrathecal space (12). In this configuration, the biosensor (160) continuously or periodically monitors the CSF without necessitating aspiration and reintroduction of the fluid. At predetermined sampling intervals, the near-infrared biosensor (160) emits NIR light into the adjacent CSF and captures the back-scattered or transmitted light using its photodetectors to obtain optical absorption characteristics corresponding to the drug concentration. The electrical signal generated by the biosensor (160) at these sampling intervals is transmitted via the dedicated sensor data lumen (140) to the central pump controller (170). The controller (170) processes the signal using established subject-specific pharmacokinetic models and compares the measured drug concentration against preset therapeutic thresholds. Based on this analysis, the closed-loop control algorithm determines whether adjustments to the drug infusion rate are necessary. Accordingly, the controller (170) sends a command to the automated valve system, which then pumps a calculated dose of the therapeutic agent from the reservoir (110a-110n) directly into the intrathecal space (12). This direct delivery methodology minimizes potential fluid shifts associated with CSF aspiration and ensures that dosing aligns precisely with the detected needs in real time. This embodiment offers the technical advantage whereby the biosensor's (160) placement at the catheter tip (132) in the CSF streamlines the monitoring process. Sampling at controlled frequencies allows for accurate, near-continuous assessment of intrathecal drug levels, thus facilitating a more responsive and fine-tuned adjustment of the infusion parameters. As such, this variation further enhances therapeutic precision while reducing the invasiveness associated with intermittent CSF handling.

In certain embodiments, the system (1000) further comprises an access port (190) having a self-sealing septum (192), and a radio opaque or wireless locator for precise location of the access port (190) for refilling the reservoir (110a-110n) and/or for sampling CSF transcutaneously, and the sensor (160) may include a near-infrared spectroscopic sensor, a temperature sensor, a pressure sensor, an environmental sensor, an osmolarity sensor, a biomarker sensor, or any combination thereof.

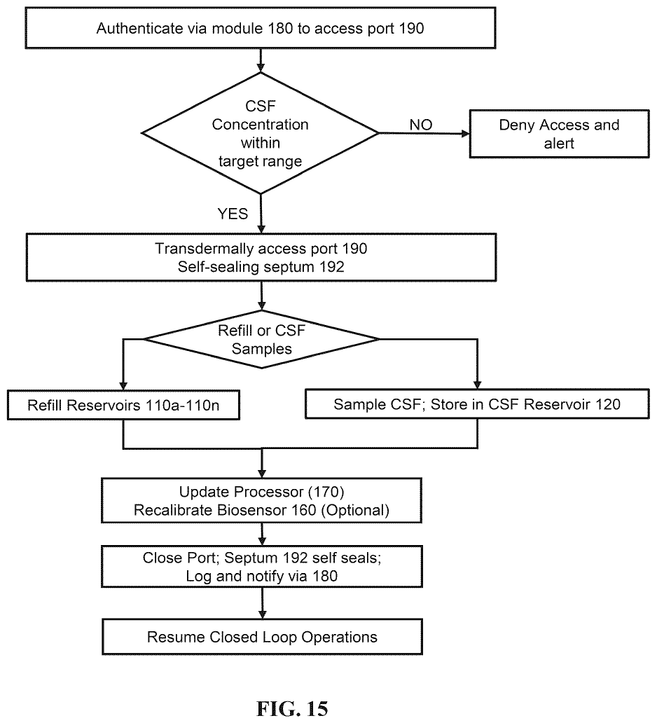

In another embodiment, the implanted system is operating in its closed-loop dosing mode when a clinician initiates a refill/diagnostic procedure, as shown in FIG. 15. First, the clinician requests access to the wireless communication module (180) and presents valid credentials (e.g., cryptographic token and PIN). The controller (170) executes the “Authenticate via module 180” step of the flow and branches at the “Authenticate Success? (Yes/No)” decision. If authentication fails (No branch), the device denies maintenance actions, maintains or transitions to a safe therapeutic state, issues a patient-perceptible alert via the alert module (206), records the failed attempt with a timestamp, and enforces a lockout interval; no refill or sampling can proceed until a subsequent successful authentication. If authentication succeeds (Yes branch), the controller transitions to a maintenance mode and enables sterile, transdermal access to the access port (190). Under aseptic technique, a non-coring needle (e.g., 22-25 G) is advanced orthogonally through the self-sealing septum (192) (“Transdermally access port 190/Septum 192”).

When the session is a refill, the controller sequences individual reservoir channels (110a-110n) in turn (“Refill Reservoirs 110a-110n”) for the selected reservoir, the associated microvalve (112a-112n) opens while downstream check-valving remains closed to prevent backflow into shared manifolds, the pump (150) is inhibited or set to a low hold state, fill rate and head pressure are monitored, and the controller terminates the fill on reaching a programmed volume (e.g., V_target±tolerance) or pressure bound. The process can be repeated for additional reservoirs, after which the controller performs a leak-down check for a short interval before exiting the refill loop.

When the session includes CSF sampling, a measured aliquot (e.g., 0.2-1.0 mL) is withdrawn through the catheter's CSF path into the CSF reservoir (120) (“Sample CSF; Store in CSF Reservoir 120”). The controller then executes “Update Processor (170)” to register the new sample metadata and, in certain implementations, initiates “Recalibrate Biosensor 160 (Optional)”: the NIR biosensor (160) is checked against the fresh CSF sample using a one-point or multi-point optical check; updated calibration constants are computed and committed to non-volatile memory, with rollback to prior coefficients if quality checks fail.

Upon completion of the desired actions, the needle is withdrawn; the septum (192) self-seals (“Close Port; Septum 192 self-seals”). The controller verifies port closure, clears the maintenance state, and records a comprehensive audit trail, including the authentication outcome, reservoirs serviced, volumes added, CSF volume/quality flags, and any calibration updates. The device then “Logs and notifies via 180” transmitting a completion report and updated parameters to an external console or cloud dashboard and “Resumes Closed-Loop Operations” at the prior or newly calibrated dose profile. Suppose any excursion (e.g., occlusion pressure, abnormal draw, authentication timeout) occurs during the session. In that case, the controller aborts the routine, returns valves to a closed condition, issues an alert, and persists diagnostic codes for clinician review.

In an alternative configuration, the NIR biosensor (160) is embedded in the housing (102), and an optical conduit runs within (140) to a clear-windowed segment near the distal tip (132). This isolates electronics from CSF yet preserves optical sampling at the point of delivery.

In further embodiments, the system may include a plurality of reservoirs (110a-110n), each configured to contain a distinct therapeutic agent, and the processor (170) may independently control the dispensing of each agent. The multi-lumen catheter (130) may include two or more lumens configured for inflow (134) and outflow (136) of CSF, and/or for simultaneous or sequential delivery of multiple drugs (138).

A temperature sensor thermally coupled to the CSF reservoir (120) provides continuous CSF temperature monitoring. When the additional sensor (162) detects T≥T_fever for a sustained interval, the processor (170) transitions to a “safe state”: pauses (150), closes (112a-112n), and actuates alert (206). On recovery to T<T_reset for Δt, the controller resumes baseline dosing.

In certain embodiments, the system (1000) may comprise haptic feedback to the pump's interface, which may provide subjects with physical alerts regarding their medication status or reminders for any required action, such as confirming dosage adjustments.

In a simplified embodiment, the catheter (130) is dual-lumen: (134) aspirates and (136) returns CSF/drug mixture; optical sensing (160) resides in the housing (102), and measurements are performed on aspirated CSF passing an in-line cuvette.

The processor (170) executes a closed-loop algorithm that integrates CSF concentration (from 160), pressure, and temperature (from 162), as well as dosing history, to compute a control signal for (150) and (112a-112n). In certain implementations, a supervised model (e.g., gradient-boosted regression or LSTM) is trained on patient-specific PK/PD sessions stored locally. Inference runs on-device, and hard safety limits and rate-of-change caps bound commands.

The wireless module (180) supports authenticated bidirectional control (e.g., BLE with rotating keys and biometric unlock). The system synchronizes dosing schedules with another implanted stimulator via time-stamped messages so that infusion peaks align with stimulation epochs.

For pediatric use, the catheter (130) features a reduced outer diameter (OD) construct with a softer durometer; anchoring sleeves and vertebral-level clips are also scaled accordingly. Reservoir volumes are reduced, with the processor (170) enforcing pediatric-specific maximum hourly dose.

From the above description, it will be appreciated that many variations are possible in the system. Although every possible embodiment has been disclosed in its preferred form(s), the specific embodiment as disclosed and illustrated herein is not to be considered in a limiting sense, because numerous variations are possible. The subject matter of the inventions includes all novel and nonobvious combinations and sub-combinations of the various elements, features, functions, and/or properties disclosed herein. The following claims particularly point out certain combinations and sub-combinations regarded as novel and nonobvious. Inventions embodied in other combinations and sub-combinations of features, functions, elements, and/or properties may be claimed in applications claiming priority from this or a related application. Such claims, whether directed to a different invention or to the same invention, and whether broader, narrower, equal, or different in scope to the original claims, are also regarded as included within the subject matter of the inventions of the present invention.

1. An implantable intrathecal drug-delivery system, comprising:
a device housing configured for subcutaneous implantation, the device housing having:
a multi-lumen catheter operatively coupled to the device housing and configured to position a distal tip within a spinal intrathecal space;
a plurality of reservoirs disposed in the device housing, each of the plurality reservoirs being configured to contain at least one therapeutic agent;
at least one pump fluidly coupled to at least one of the plurality of reservoirs and to a drug delivery lumen of the multi-lumen catheter;
at least one cerebrospinal-fluid (CSF) reservoir disposed in the device housing and configured to receive and store CSF sampled from the spinal intrathecal space;
at least one biosensor comprising a near-infrared (NIR) spectroscopic sensor positioned at or near the distal tip of the multi-lumen catheter, or optically coupled to the distal tip, the biosensor being disposed in the catheter and/or in the device housing, the biosensor configured to obtain, from CSF, a sensor signal indicative of a concentration of at least one therapeutic agent;
wherein the multi-lumen catheter comprises fluid-isolated lumens including:
(i) a CSF inflow lumen configured to direct CSF from the spinal intrathecal space into the device housing;
(ii) a CSF return lumen configured to direct CSF from the device housing to the spinal intrathecal space;
(iii) the drug-delivery lumen configured to deliver the therapeutic agent from the device housing to the spinal intrathecal space; and
(iv) a power/data lumen configured to provide power and/or data transmission for the biosensor;
a processor operatively coupled to the biosensor, pump, and the plurality of reservoirs, the processor being configured to receive biosensor-derived concentration data based on the sensor signal determine, in real time, the concentration of the at least one therapeutic agent in the CSF, compare the concentration with a predefined therapeutic concentration range, and adjust an infusion rate of the therapeutic agent through the drug-delivery lumen based on the comparison while maintaining separation between CSF inflow/return and drug delivery;
a wireless communication module operatively coupled to the processor and configured for bidirectional communication with an external device; and
at least one transdermal access port in the device housing, the access port comprising a self-sealing septum and configured to permit reservoir refilling and CSF sampling.

2. The system of claim 1, further comprising at least one additional biosensor selected from a temperature sensor, a pressure sensor, an osmolarity sensor, a biomarker sensor, or an environmental sensor.

3. The system of claim 2, wherein the NIR spectroscopic sensor is configured for in-situ CSF monitoring.

4. The system of claim 3, wherein the NIR spectroscopic sensor is positioned at or near the distal tip of the catheter to directly sense the concentration of the therapeutic agent within the intrathecal space.

5. The system of claim 1, wherein the at least one additional sensor is configured to provide real time data to the processor for adjusting one or more therapy-delivery parameters, inhibiting the pump, modulating dosage, or providing a safety fallback response.

6. The system of claim 1, wherein the wireless communication module implements multi-factor authentication selected from biometric identification, radio-frequency identification (RFID) authentication, or cryptographic keys.

7. (canceled)

8. The system of claim 7, wherein the power/data lumen is configured to provide power and/or data transmission for the at least one biosensor.

9. The system of claim 1, wherein the processor is configured to wirelessly synchronize infusion parameters with at least one additional implanted device or an external therapeutic-management platform.

10. The system of claim 1, wherein the device housing further comprises at least one patient-alert module selected from visual, auditory, or haptic feedback devices configured to notify a patient of therapeutic-administration events or device malfunctions.

11. The system of claim 1, wherein microfluidic channels integrated within the catheter and/or the device housing are configured to enable controlled intrathecal delivery of an advanced therapeutic agent selected from nanoparticle-based drugs, gene therapies, viral vectors, antibodies, or nanorobotic agents.

12. The system of claim 1, wherein the plurality of reservoirs include individually controllable microvalves responsive to the processor (170) to selectively dispense two or more of the at least one therapeutic agents simultaneously or sequentially.

13. The system of claim 1, further comprising an auxiliary housing implanted subcutaneously and operatively linked via a common multi-lumen catheter to the device housing.

14. A method for intrathecal delivery of a therapeutic agent, comprising:
implanting the intrathecal drug-delivery system of claim 1 with the device housing subcutaneously and positioning the multi-lumen catheter such that the distal tip resides within the spinal intrathecal space;
directing CSF from the spinal intrathecal space to the CSF reservoir through the CSF inflow lumen;
directing CSF from the device housing back to the spinal intrathecal space through the CSF return lumen;
delivering the therapeutic agent from at least one of the plurality of reservoirs to the spinal intrathecal space through the drug-delivery lumen;
obtaining, continuously or periodically, by the biosensor, a sensor signal from CSF in the spinal intrathecal space, the sensor signal being indicative of a concentration of the therapeutic agent;
transmitting, to the processor, biosensor-derived concentration data based on the sensor signal through the power/data lumen;
determining, by the processor in real time, the concentration of the therapeutic agent from the biosensor-derived concentration data;
comparing, by the processor, the concentration with a predefined therapeutic concentration range; and
adjusting, by the processor, the infusion rate of the therapeutic agent based on the comparison while maintaining fluidic separation between CSF inflow/return and drug delivery.

15. The method of claim 14, further comprising wirelessly transmitting the biosensor-derived concentration data and infusion parameters to an external device via the wireless communication module.

16. The method of claim 14, further comprising transdermally refilling one or more of the reservoirs via the access port integrated into the device housing.

17. The method of claim 16, further comprising localizing the access port using a radiopaque marker and/or a wireless locator prior to refilling, and enabling refilling only after successful authentication via the wireless communication module.

18. The method of claim 14, further comprising sampling CSF via the access port for diagnostic evaluation, therapeutic monitoring, or calibration of patient-specific infusion parameters.

19. The method of claim 14, further comprising synchronizing therapeutic-agent delivery with at least one additional implanted device or an external therapeutic-management platform.

20. The method of claim 14, further comprising implanting an auxiliary housing subcutaneously and operatively linking the auxiliary housing to the device housing via a common multi-lumen catheter.

21. The method of claim 20, wherein transdermally refilling comprises refilling a reservoir in the auxiliary housing via the access port and a self-sealing septum, or via a dedicated service port.

22. The system of claim 1, wherein the fluid-isolated lumens are separated by internal septa to minimize cross-communication between CSF handling, drug delivery, and biosensor power/data transmission.
